(19) United States
(12) Patent Application Publication
(10) Pub. No.: US 2004/0106988 A1
(43) Pub. Date: Jun. 3, 2004

(54) Resorbable prosthesis for medical treatment

(76) Inventor: David P. Summers, Montgomery, TX (US)

Correspondence Address: NICK A NICHOLS, P O BOX 16399, SUGARLAND, TX, 774966399

(21) Appl. No.: 10/381,873

(22) PCT Filed: Sep. 28, 2001
(86) PCT No.: PCT/US01/30409

(30) Foreign Application Priority Data
Sep. 29, 2000 (US) 60236593

(51) Int. Cl. A61F 2/06

(52) U.S. Cl. 623/1.42; 604/508; 424/426

(57) ABSTRACT

A resorbable, biodegradable, drug delivery prosthesis (10) includes mechanical properties for maintaining the strength needed to acutely open and maintain a vessel, duct, tract, or organ, and precise chronicity for controlling the release and delivery of drugs or biologic agents. The drugs and biologic agents are capable of acting upon and altering the mechanisms of biologic systems in an manner providing a medicinal therapy. The method of the invention includes a one step process of extruding the admixture forming the prosthesis (10) into a geometry compatible with its use.

### BACKGROUND OF THE DISCLOSURE

The present invention relates generally to the field of localized stent treatment; more specifically to the use of resorbable prostheses with biodegradable surface coating incorporating drugs or bioactive agents for treating a medical condition.

The use of implantable medical devices to treat a variety of medical conditions by introducing the device into a body cavity, tract, duct or vessel has become common medical practice. Treatment of blood vascular disease such as occlusions, obstructions, and stenoses of the blood vessels resulting from atherosclerosis—a disease of atherosclerotic plaques and cholesterol deposits—routinely employ the use of small metal scaffolds called intravascular stents to ameliorate the ischemic condition caused by these blockages. In a more detailed description, the use of stents is described in U.S. Pat. No. 5,824,649 and more particularly in U.S. Pat. No. 5,980,551 which incorporates the use of a biodegradable substrate which is loaded with a drug or active agent to chronically release said drug or active agent from a placement site within a mammalian body. The above invention of the '551 patent sought to accomplish the opening and maintenance of the opening of a blood vessel by mechanical means while providing medicinal drug treatment from the gradual release of such drugs from the slowly degrading biocompatible substrate of the intravascular stent. Such stents are capable of chronic release of various drugs from a period of a few days to a period of many months.

Many drugs are useful for incorporation into biodegradable and thus, bioactive prosthetic stents. In the case of U.S. Pat. No. 5,980,551, a poly-L-lactic (PLLA)/Poly-caprolactone (PCL) copolymer blend of aliphatic polyester has proven both degradable, resorbable and hemo-compatible. Depending on the ratio of PLLA/PCL, these coatings can provide a benign substrate that provides a microporous structure that can efficiently be impregnated with biologically active drugs either bound, associated, conjugated with the substrate, or impregnating the substrate with biologically active microspheres or liposomes in the range of 20 nm to 1000 nm diameter.

One method of making a stent is to extrude a thin-walled tubular member, such as resembling a metal hypotube, but with materials resistant to chemical etchants. When material other than the etchants are removed from the tube the remaining portion forms the prosthesis. Such method is described more fully in European Patent Specification, EPO6793 73B1, to Dixon et al. Another method is described in U.S. Pat. No. 5,824,049, to Ragheb, et al. However, both patents describe a method for coating an implantable metal device and only the substrates are biodegradable, leaving the metal body as a permanent implant. Additionally, coating the metal body requires substantial cleaning, passivation, acid treatment, and control of electrostatic charge, none of which is required by the instant invention.

It is therefore another object of the invention to provide a prosthesis, such as a vascular stent which is biocompatible, biodegradable and resorbable to provide precise local delivery of undiluted drugs, agents or bioactive agents directly into diseased organs, tissues, systems, circuits, or networks (such as neural networks). This objective is accomplished by placing a prosthesis, such as a vascular stent, directly into said organ, tissues, system, circuit or network, or directly proximal to such site (e.g., a feeding artery of a tumor) to treat said disease, and controlling the dosing from said prosthesis over a period of weeks to months.

### SUMMARY OF THE INVENTION

The present invention comprises a method of making a totally resorbable, biodegradable, drug delivery prosthesis having mechanical properties for maintaining the strength needed to acutely open and maintain a vessel, duct, tract, or organ, but having precise chronicity controlling the release and delivery of drugs or biologic agents. The drugs and biologic agents are capable of acting upon and altering the mechanisms of biologic systems in a manner providing a medicinal therapy. The method of the present invention includes a one step process of extruding all of the admixture forming the prosthesis into a geometry compatible with its use.

### BRIEF DESCRIPTION OF THE DRAWINGS

So that the manner in which the above recited features, advantages and objects of the present invention are attained can be understood in detail, a more particular description of the invention briefly summarized above, may be had by reference to the embodiments thereof which are illustrated in the appended drawings.

It is noted, however, that the appended drawings illustrate only typical embodiments of this invention and are therefore not to be considered limiting of its scope, for the invention may admit to other equally effective embodiments.

FIG. 1 is perspective view of the prosthetic device of the invention.

### DETAILED DESCRIPTION OF A PREFERRED EMBODIMENT

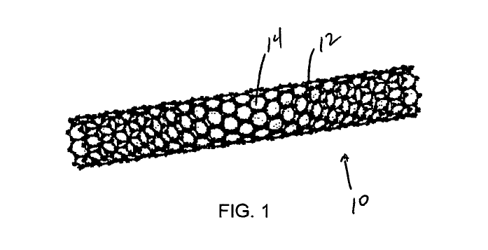

In a preferred embodiment of the invention, a thin-walled tube of resorbable, biodegradable polymer is extruded. The thin-walled polymer tube is then etched by a laser etching process well known in the art, leaving a pre-determined pattern of various geometric lattice, arcuate coils, helixes and double helixes as described in U.S. Pat. Nos. 5,607,445, 5,772,668, and 6,080,191, to Summers, which are incorporated by reference herein. Referring now specifically to FIG. 1 a stent prosthesis of the invention, generally identified by the reference numeral 10, is shown. The prosthesis 10 comprises a substantially tubular body 12 open at both ends. The stent body 12 is formed of resorbable polymers so that it degrades over time and is resorbed by the surrounding tissue, for example, a vessel wall. The stent body 12 may be formed by an extrusion process or alternatively may be cast. The stent body 12 is laser etched to form a plurality of openings 14 throughout the stent body 12 resulting in a mesh like appearance. In the present invention, problems associated with coating an etched prosthesis body with biodegradable polymers are overcome. The extruded tube of the present invention comprises an admixture of resorbable, biodegradable polymers containing at least one drug, which is loaded into a parent polymer or into a microsphere or liposome prior to extrusion. The drug or microsphere or liposome containing drug, is then cross-linked, covalently bound, conjugated, derivatized, or associated with the parent polymer during mixing or during the extrusion step, and thereby eliminating the extra steps of coating the prosthetic body after it is formed. The prosthetic body formed by the process of the invention has sufficient mechanical strength to support the surrounding tissue of a blood vessel, duct or the like.

The prosthesis 10 in FIG. 1 is depicted as having a tubular body for illustrative purposes. It is understood that the body of the prosthesis 10 may comprise various configurations suitable for supporting a vessel in an open condition.

In a preferred embodiment of the present invention, the active agent encapsulated within the admixture of resorbable, biodegradable polymers may, for example, be prostaglandin E1 (PGE1), a naturally occurring fatty acid of the cyclopentenone family. The timed release of PGE1 produces powerful chronic antagonistic chemotaxis to thromboxane and leukotrience actions on the platelets and injured vessel wall while modulating the proliferation of smooth muscle cells (SMC) and extracellular matrix within the media of the blood vessel, duct or the like. This two-stage process continues to produce inhibition of protein absorption and hence cellular interactions at the bio-material surface, while releasing powerful inhibitions of platelet aggrandizement and modulators of cell growth in the region of the vessel where the stent is located. The protein inhibiting action of the biologically active agent continues over a predetermined period of weeks or months or until endothelialization of the bio-surface is complete. Of particular note, the labile PEG end-groups on these modified surfaces may be made to serve as attachment sites for suitable bio-specific peptides, that results in a surface that may potentially adhere to only one particular cell type, such as endothelial cells, in the case of stent or vascular grafts.

The preferred embodiment of the invention may include the delivery of anticancer, antiproliferative, preoperative tumor debulkers or chemotherapeutic agents directly to a tumor. It should also be noted that pallatives which ease the symptoms of the disease such as anesthetics, analgesics, neural stimulators, agonists and antagonist are also included in the scope of the invention. In the case of pallatives, for example, procaine or morphine may be administered for pain control. Nicotine or nicotine receptor agonist may be placed in the vascular supply of the thalamic substantia nigra for treatment of neurodegenerative disease such as Alzheimer's, Parkinson's, Huntington's and Lou Gehrig's disease.

In the case of antimototics, antiproliferative, antisecretor, growth factors and antigrowth factor agents, thalidomide, taxol, and cisplatin are useful examples. Antisense oligomers, basic fibroblast growth factor, vascular endothelial growth factors or antagonist of growth factors, cell migration, cell differentiation and replicating chemokines, synthases or cell signaling factors are disclosed.

Immunosuppressive agents, such as cyclosporin, may be utilized in the present invention to provide long-term immunosuppressive therapies. In the case of organ or tissue transplant therapies, hormones such as testosterone, estrogen for steroid deficiencies, dexamethasone and various prostaglandins for inflammatory therapies are also contemplated for use in the instant invention.

One method of making a stent in accordance with the invention is a casting process wherein the polymer mixture is cast into its final form, thus eliminating the additional step of coating the prosthetic body with therapeutic drugs, bioactive agents, bioactive materials or microspheres and liposomes containing such agents.

Accordingly, it is desirable to control the chronicity of the drug treatment over short term regimens (hours and days) or longer term (weeks and months). It is also desirable that the medicines delivered from the prostheses of the invention be subject to precise control over the delivery rate for drugs, agents, or bioactive material, and to limit the systemic exposure to them. In such a case, at least two layers or different substrate combinations may be utilized in the casting process forming the stent prostheses, each substrate having a degradation rate dissimilar from the other. Each substrate may be further controlled in the release of drugs or bioactive agents by embedding in each substrate dissolvable microspheres or liposomes having a dissolution rate independent of the substrate.

This would be particularly advantageous in therapies involving the delivery of chemotherapeutic agents to a particular organ, reducing the systemic amount but increasing that local amount of the agent for successful local treatment which also avoids degradation of the agent, drug or bioactive material.

While a preferred embodiment of the invention has been shown and described, other and further embodiments of the invention may be devised without departing from the basic scope thereof, and the scope thereof is determined by the claims which follow.

1. A prosthetic device comprising:
a) a resorbable biodegradable body; and
b) wherein said resorbable body comprises a mixture of resorbable, biodegradable polymers and at least one pharmaceutically active agent.

2. The prosthetic device of claim 1 wherein said resorbable body is formed by an extrusion process as a single unitary structure.

3. The prosthetic device of claim 1 wherein said resorbable body is formed by a casting process as single unitary structure.

4. The prosthetic device of claim 3 wherein said resorbable body includes one or more layers of a resorbable mixture encapsulating a pharmaceutically active agent.

5. The prosthetic device of claim 1 wherein release of said at least one pharmaceutically active agent is chronically controlled.

6. The prosthetic device of claim 1 wherein said at least one pharmaceutically active agent is prostaglandin E1.

7. The prosthetic device of claim 1 wherein said mixture includes a bioactive molecule or microsphere or liposome encapsulating a pharmaceutically active agent or drug.

8. The prosthetic device of claim 1 wherein said at least one pharmaceutically active agent is a nicotine receptor agonist.

9. The prosthetic device of claim 8 wherein said nicotine receptor agonist is nicotine.

10. The prosthetic device of claim 1 wherein said mixture of resorbable biodegradable polymers includes biologically active microspheres, liposomes, bound drugs, associated drugs, derivatized drugs and conjugated drugs.

11. A stent prosthesis comprising:
a) a resorbable biodegradable body;
b) wherein said resorbable body comprises a mixture of resorbable, biodegradable polymers and at least one pharmaceutically active agent; and
c) wherein said resorbable body is formed by an extrusion process as a single unitary structure.

12. The stent prosthesis of claim 11 wherein release of said at least one pharmaceutically active agent is chronically controlled.

13. The stent prosthesis of claim 11 wherein said at least one pharmaceutically active agent is a nicotine receptor agonist.

14. The stent prosthesis of claim 13 wherein said nicotine receptor agonist is nicotine.

15. The stent prosthesis of claim 11 wherein said mixture of resorbable biodegradable polymers includes biologically active microspheres, liposomes, bound drugs, associated drugs, derivatized drugs and conjugated drugs.

16. A method for treating a medical condition comprising:
a) placing a resorbable biodegradable prosthesis in a body cavity, tract, duct or vessel;
b) wherein said resorbable prosthesis comprises a mixture of resorbable, biodegradable polymers and at least one pharmaceutically active agent formed as a single unitary structure; and
c) chronically controlling the release rate of said pharmaceutically active agent.

17. The method of claim 16 wherein said at least one pharmaceutically active agent is prostaglandin E1.

18. The method of claim 16 wherein said at least one pharmaceutically active agent is a nicotine receptor agonist.

19. The method of claim 16 including the step of encapsulating, bounding, associating, derivatizing or conjugating said at least one pharmaceutically active agent with said resorbable prosthesis.

20. The method of claim 16 including the step of delivering high localized drug titers to by controlling the release of microspheres or liposomes encapsulating said drug.
